(19) United States
(12) Patent Application Publication
(10) Pub. No.: US 2025/0268747 A1
(43) Pub. Date: Aug. 28, 2025

(54) Adjustable Therapy Wrap

(71) Applicant: Daniel H. Godfrey, Newberg, OR (US)

(72) Inventor: Daniel H. Godfrey, Newberg, OR (US)

(21) Appl. No.: 18/589,249

(22) Filed: Feb. 27, 2024

(51) Int. Cl. A61F 7/02

(52) U.S. Cl. CPC A61F 7/02; A61F 2007/0219; A61F 2007/0231; A61F 2007/0234; A61F 2007/0236; A61F 2007/0277

(57) ABSTRACT

A therapy wrap that treats pain and swelling by applying compression and hot or cold therapy to the afflicted part of the body. In some implementations, the device may consist of a right-hand piece with an outer and inner surface. Additionally, the device may have a first end and a second end. The device may also include a fastening mechanism on the inner surfaces of both ends. Furthermore, the device may have a left-hand piece with an outer and inner surface. It may also have a first end and a second end, as well as a fastening mechanism located on surface of both ends. The right-and left-hand pieces may be joined together or used separately. Either piece can apply a therapy gel pack to the afflicted area.

### BACKGROUND

The present disclosure relates to treating pain and swelling using compression, hot, or cold therapy. Swelling, strains, sprains, and pain are natural parts of life. They are common and have many causes, such as injuries from sports, exercise, accidents, or disease. The use of compression, cold, and heat to the affected area is a generally accepted course of treatment. Thus, there exists a need to apply the treatment to the affected area easily. The invention disclosed herein facilitates applying said treatment to the affected body area, such as the torso, ankles, elbows, wrists, shoulders, upper back, or lower back.

### SUMMARY

The invention is an adjustable therapy wrap that can be separated into two pieces. Separation allows the wrap to be configured to the affected part of the body. For example, when joined together, the wrap will fit around the torso (FIGS. 4 and 5). When separated, the wrap can be fit around smaller body parts such as the ankle (FIG. 6) or the elbow (FIG. 7). The invention provides convenience to the user because it can treat multiple affected areas of the body. The wrap facilitates treatment by applying pressure and adding therapy gel packs (FIG. 3). The wrap holds the packs against the affected area. The gel packs can be heated or cooled depending on the recommended course of treatment.

### BRIEF DESCRIPTION OF THE DRAWINGS

FIG. 1 shows both pieces of the wrap joined together.

FIG. 2 shows the wrap as separate pieces.

FIG. 3 shows how the wrap can be used with therapy gel packs.

FIG. 4 shows a front view of the joined wrap in service.

FIG. 5 shows a rear view of the joined wrap in service.

FIG. 6 shows one piece of the wrap in service around an ankle

FIG. 7 shows one piece of the wrap in service around an elbow.

### DETAILED DESCRIPTION

The disclosed invention will become better understood by reviewing the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for brevity, every contemplated variation is not individually described in the following detailed description.

Throughout the following detailed description, examples of various elements are provided. Related features in the examples may be identical, similar, or dissimilar in different examples. For the sake of brevity, related features will not be redundantly explained in each example. Instead, using related feature names will cue the reader that the feature with a related name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein unless otherwise indicated.

“Comprising,” “including,” and “having” (and conjugations thereof) are used interchangeably to mean including but not necessarily limited to and are open-ended terms not intended to exclude additional elements or method steps not expressly recited.

Terms such as “first,” “second,” and “third” are used to distinguish or identify various members of a group or the like and are not intended to denote a serial, chronological, or numerical limitation.

### Adjustable Therapy Wrap

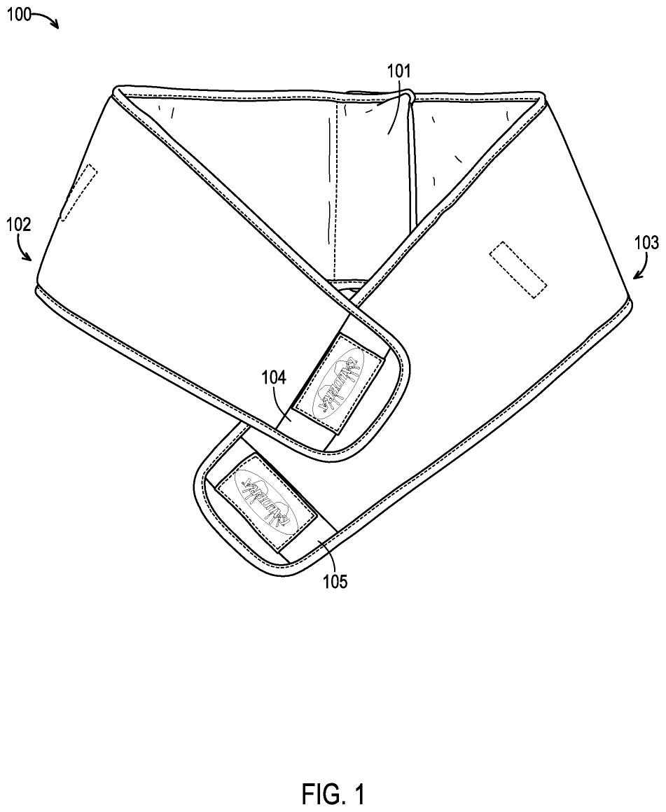

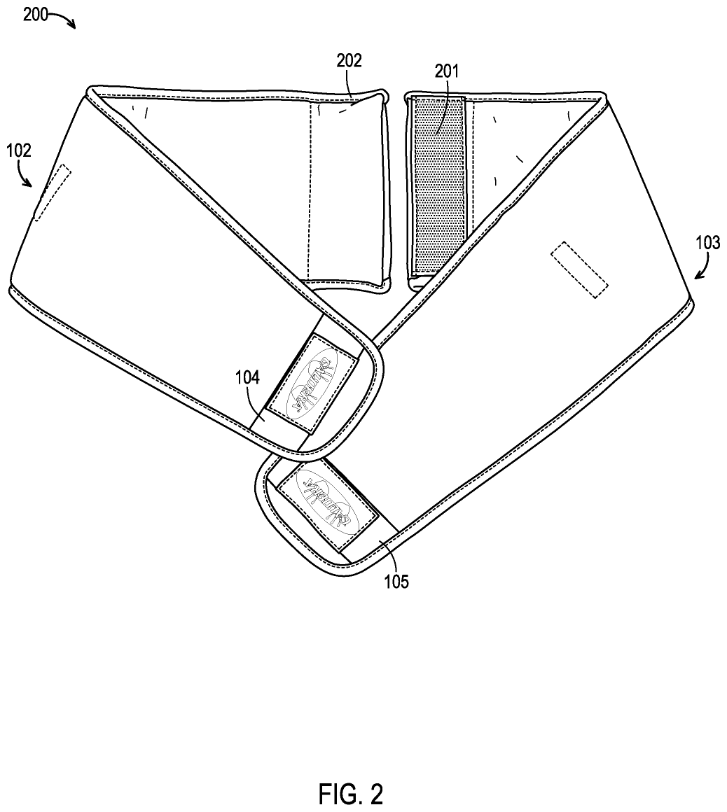

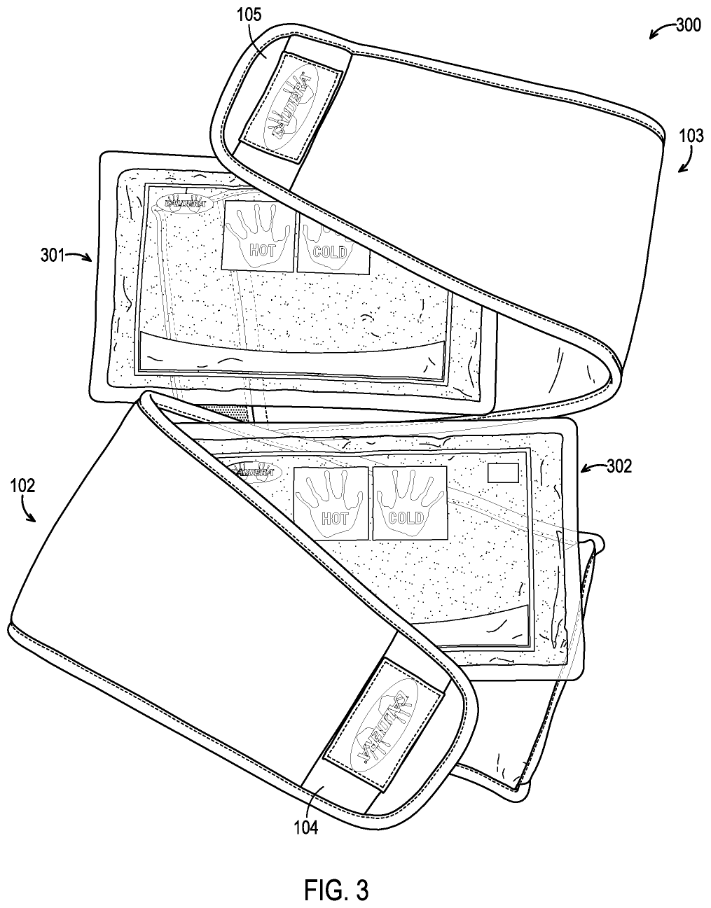

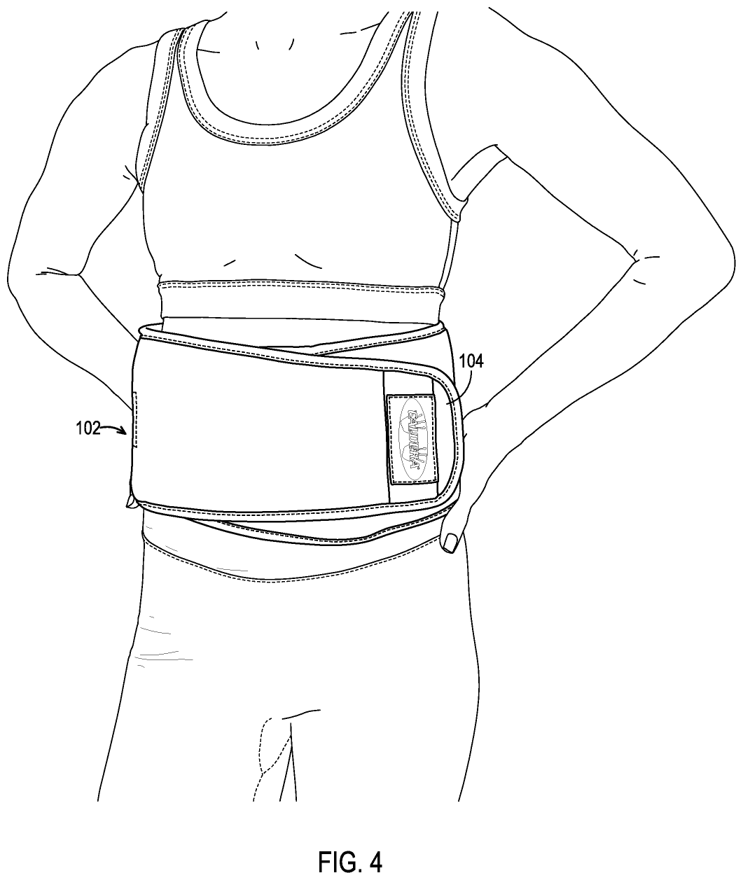

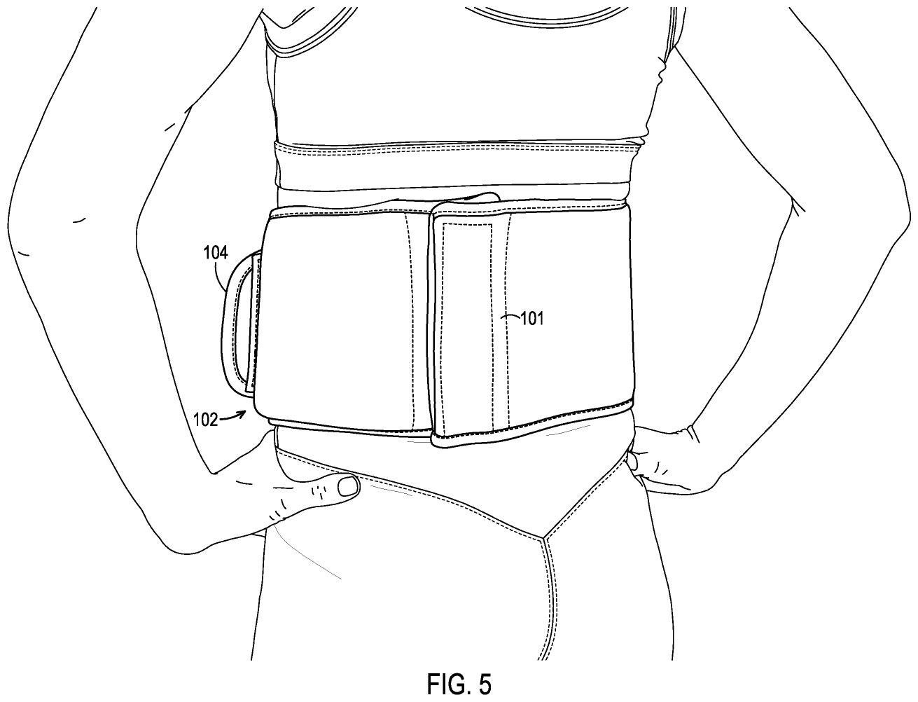

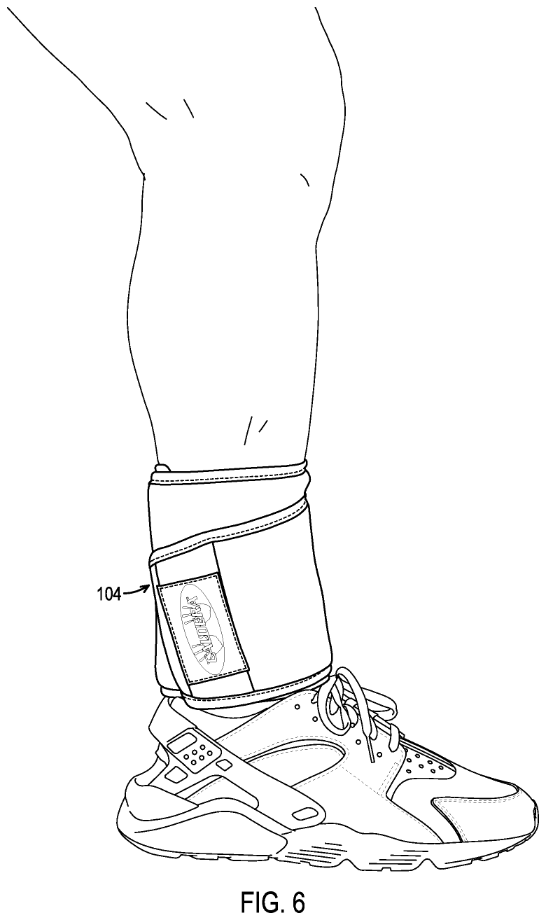

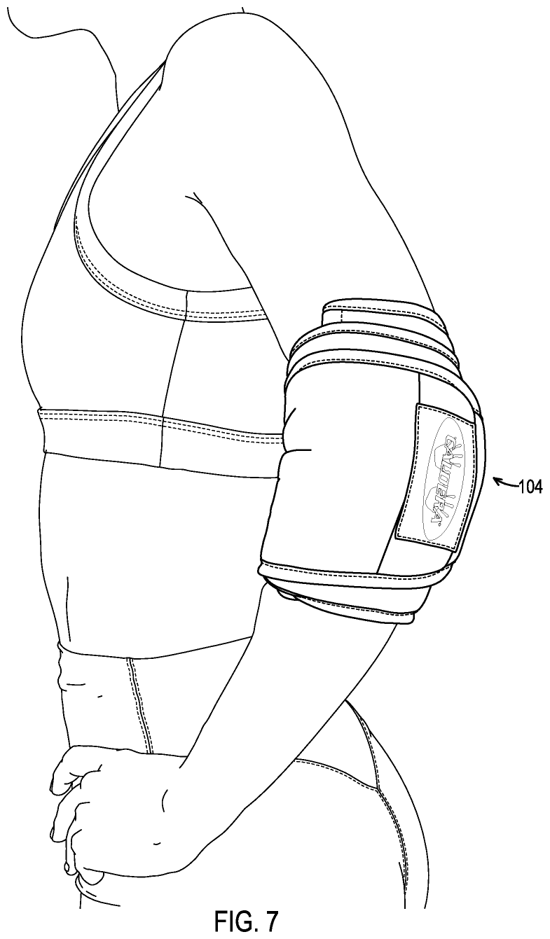

With reference to FIGS. 1 to 7, the Adjustable Therapy Wrap will now be described. The adjustable therapy wrap 100 comprises two pieces (102 and 103) that are releasably fastened together 101. Each piece (102 and 103) is a righthanded 103 and lefthanded 102 pair. Each piece has a first and second end, where righthand 103 has a first end 105 and a second end 201; similarly, the lefthand piece 102 has a first end 104 and a second end 202.

The wrap further comprises a fastener that reversibly holds the righthand piece 103 and lefthand piece 102 together. The two pieces are joined at their respective second ends, 201 and 202. At the respective first ends, 104 and 105, another fastener reversibly holds the wrap closed when put in service. Non-limiting examples of fasteners include any hook and loop combination, buttons, snaps, zippers, or safety pins.

In the preferred embodiment, the fastener is a hook-and-loop system. At the second end, the hook portion of the fastener is attached to one of the pieces, and the loop portion is attached to the second end of the other piece. A hook fastener is attached to the first end of each piece. The outer surface of the wrap, which does not come in contact with the body, provides the loop to which the hooks attach. In another embodiment, the wrap holds packets of therapy gel (301 and 302) against the desired part of the body. In a third embodiment, when joined together, the wrap becomes a back therapy wraps. Lastly, in fourth embodiment, when joined together, the wrap can be used to provide lumbar support.

Applicant(s) reserves the right to submit claims directed to combinations and subcombinations of the disclosed inventions that are believed to be novel and non-obvious. Inventions embodied in other combinations and sub-combinations of features, functions, elements, and/or properties may be claimed through amendment of those claims or presentation of new claims in the present application or a related application. Such amended or new claims, whether directed to the same invention or a different invention and whether they are different, broader, narrower, or equal in scope to the original claims, are to be considered within the subject matter of the inventions described herein.

1. An adjustable therapy wrap comprising: at least one piece having an outer surface and, an inner surface; and a first end and a second end; and a fastening means disposed on the inner surface of the first end and the inner surface of the second end.

2. The adjustable therapy wrap of claim 1, further comprising at least one packet of therapy gel disposed on the inner surface of the adjustable therapy wrap.

3. The adjustable therapy wrap of claim 2, wherein the fastening means is a hook and loop fastener.

4. The adjustable therapy wrap of claim 2, wherein the fastening means further comprises snaps.

5. The adjustable therapy wrap of claim 2, wherein the fastening means comprises buttons.

6. The adjustable therapy wrap of claim 2, wherein the fastening means is a zipper.

7. An adjustable therapy wrap comprising: a righthand piece having an outer surface and an inner surface; a first end and a second end; and a fastening means disposed on the inner surface of the first end and on the inner surface of the second end; and a lefthand piece having an outer surface and an inner surface; a first end and a second end; and a fastening means disposed on the inner surface of the first end and on the outer surface of the second end.

8. The adjustable therapy wrap of claim 7, where the fastening means is a hook and loop system.

9. The adjustable therapy wrap of claim 8, the righthand and lefthand pieces are fastened together at their respective second ends.

10. The adjustable therapy wrap of claim 9, where the outer surface is one part of the hook and loop fastening means.

11. The adjustable therapy wrap of claim 10, further comprising at least one packet of therapy gel disposed on the inner surface of the adjustable therapy wrap.

12. The adjustable therapy wrap of claim 11, wherein the adjustable therapy wrap is wrapped around the lower torso, upper torso, right shoulder, left shoulder, upper back, or lower back.

13. The adjustable therapy wrap of claim 8, where the righthand piece or lefthand piece wrapped around at least one of the following: right foot, left foot, right ankle, left ankle, right knee, left knee, right elbow, or left elbow.

14. The adjustable therapy wrap of claim 13, further comprising at least one therapy gel pack disposed on the inner surface of the right-hand piece or left-hand piece.

15. A method for applying compression therapy to an afflicted part of the human body by wrapping the adjustable therapy wrap of claim 1 around the afflicted part of the body.

16. A method for applying compression and hot or cold therapy to an afflicted part of the human body by wrapping the adjustable therapy wrap of claim 2 around the afflicted part of the body.

17. A method for providing lower back therapy by wrapping the adjustable therapy wrap of claim 9 around the lower part of the torso.

18. A method for providing lumbar support by wrapping the adjustable therapy wrap of claim 9 where the therapy wrap is wrapped around the torso.

19. A method for applying compression therapy to an afflicted part of the human body by wrapping the adjustable therapy wrap of claim 10 around the afflicted part of the body.

20. A method for applying compression and hot or cold therapy to an afflicted part of the human body by wrapping the adjustable therapy wrap of claim 11 around the afflicted part of the body.
